(19) United States
(12) Patent Application Publication
(10) Pub. No.: US 2024/0279814 A1
(43) Pub. Date: Aug. 22, 2024

(54) APPARATUS FOR MEASURING PLATING DEPOSITION STATUS

(71) Applicant: C. Uyemura & Co., Ltd., Osaka (JP)

(72) Inventors: Hisamitsu YAMAMOTO, Osaka (JP); Tomoharu NAKAYAMA, Osaka (JP)

(21) Appl. No.: 18/404,207

(22) Filed: Jan. 4, 2024

(30) Foreign Application Priority Data
Feb. 22, 2023 (JP) 2023-025796

(51) Int. Cl. C23C 18/16; C23C 18/32; C23C 18/38; G01K 3/14; G01N 21/31

(52) U.S. Cl. CPC C23C 18/1683; C23C 18/1628; C23C 18/1637; C23C 18/168; C23C 18/1689; C23C 18/32; C23C 18/38; G01K 3/14; G01N 21/31

(57) ABSTRACT

The present invention provides an apparatus capable of measuring a deposition status, such as a plating deposition rate, even during a plating process in a plating tank. A controller 20 controls a plating solution introduction mechanism 6 to introduce a plating solution in a plating tank 2 into a reaction container 4. By the introduced plating solution, a metal film is deposited on a deposition member 12 provided in the reaction container 4. Next, metal film dissolution control means 24 of the controller 20 controls an etching solution introduction mechanism 8 to introduce an etching solution into the reaction container 4 instead of the plating solution. The introduced etching solution dissolves the metal film deposited on the deposition member 12. A measurement equipment 10 measures a metal concentration-related value (metal concentration or a metal concentration-related value) of the post-processing etching solution in which the metal film is dissolved. Estimation means 26 of the controller 20 estimates the deposition status, such as the plating deposition rate, in the plating tank 2 on the basis of the metal concentration-related value measured by the measurement equipment 10.

### BACKGROUND OF THE INVENTION

### 1. Technical Field

The present invention relates to an apparatus for measuring a plating deposition status.

### 2. Related Art

When performing electroless metal plating, concentration of metal ions in a plating solution, a temperature of the plating solution, a plating time, and the like are adjusted according to a plating deposition status, so as to be able to perform the desired plating. For this purpose, it is important to accurately and promptly measure the deposition status, such as a deposition rate, of plated metal.

The following apparatus is disclosed in Japanese Unexamined Patent Application Publication No. S61-276978 (JP Sho 61-276978 A). The apparatus collects a sample of a plating solution from a plating tank and analyzes additives and the like contained in the collected plating solution with an analyzer. In this way, it is possible to measure a property of the plating solution that affects the deposition rate of the plated metal.

The following method is disclosed in Japanese Unexamined Patent Application Publication No. H3-240968 (JP Hei 3-240968 A). In the method, a platinum plate is immersed in a plating solution in a plating tank to deposit copper through an electroless copper plating reaction. Next, the platinum plate, on which copper is deposited, is used as an anode, and a copper plate is used as a cathode to dissolve deposited copper with a constant current. Then, a plating rate is measured by a time until complete dissolution. In this way, a plating deposition rate can be measured.

### SUMMARY OF THE INVENTION

In the related art as disclosed in JP Sho 61-276978 A, it is possible to obtain the property of the plating solution as a factor affecting the deposition rate of the plated metal, and the like. However, it is difficult to accurately estimate the deposition status such as the deposition rate. This is because the deposition rate and the like are affected by the factor other than the property of the plating solution.

In the related art as disclosed in JP Hei 3-240968 A, the deposition rate of the plated metal can be measured. However, since the platinum plate and the copper plate are immersed in the plating tank, and the current is applied thereto, there may be an adverse effect on a plating process that is actually performed in the plating tank.

An object of the present invention is to solve the problems as described above and to provide a technique capable of measuring a deposition status such as a plating deposition rate even during a plating process in a plating tank.

Another object and the like of the present invention will become apparent with reference to an embodiment and the drawings.

Some independently applicable characteristics of the present invention will be listed below. It is not intended to say that the characteristics must be combined to be the characteristics of the invention.
(1) A deposition status estimation apparatus according to the present invention is a deposition status estimation apparatus including: a reaction container having a deposition member; a plating solution introduction mechanism that introduces a plating solution in a plating tank into the reaction container in order to immerse the deposition member in the plating solution and deposit a metal solution introduction mechanism that introduces an etching solution into the reaction container in order to immerse the deposition member in the etching solution and dissolve the metal film; a measurement equipment that measures a metal concentration-related value of the etching solution that has dissolved the metal film; and a controller that executes control for measurement, in which
the controller controls to: introduce the plating solution into the reaction container by the plating solution introduction mechanism to deposit the metal film on the deposition member; introduce the etching solution into the reaction container by the etching solution introduction mechanism after the plating solution in the reaction container is discharged, so as to dissolve the metal film deposited on the deposition member; and use the measurement equipment to measure the metal concentration-related value of the etching solution, which has dissolved the metal film, and estimates a deposition status of the metal film in the plating tank on the basis of the metal concentration-related value.

Therefore, the deposition status can be estimated in real time even during processing in the plating tank.
(2) In the deposition status estimation apparatus according to the present invention, when the metal film is deposited on the deposition member, the controller executes control to discharge the plating solution from the reaction container while introducing the plating solution in the plating tank into the reaction container.

Therefore, the new plating solution is constantly supplied to the deposition member, and the metal can be deposited stably.
(3) In the deposition status estimation apparatus according to the present invention, when the metal film is deposited on the deposition member, the controller executes control to introduce the plating solution in the plating tank into the reaction container and stop the introduction once the deposition member is immersed.

Therefore, a solution level of the plating solution is stabilized, and the metal can be deposited stably.
(4) In the deposition status estimation apparatus according to the present invention, the controller estimates a deposition rate in the plating tank as the deposition status.

Therefore, the deposition rate can be estimated.
(5) In the deposition status estimation apparatus according to the present invention, the controller estimates the deposition status of the metal film in the plating tank in consideration of a difference between a temperature of the plating solution in the plating tank and a temperature of the plating solution in the reaction container, each of which is recorded or measured by a sensor.

Therefore, the deposition status of the plating tank can be estimated further accurately.
(6) In the deposition status estimation apparatus according to the present invention, the reaction container includes a heater that brings a temperature of the plating solution introduced into the reaction container close to a temperature of the plating solution in the plating tank.

Therefore, the deposition status of the plating tank can be estimated further accurately.
(7) In the deposition status estimation apparatus according to the present invention, the measurement equipment is provided in a path, through which the etching solution, which has dissolved the metal film, is discharged from the reaction container.

Therefore, the measurement can be taken while the etching solution is discharged.
(8) In the deposition status estimation apparatus according to the present invention, the metal film is copper or nickel, and the deposition member is gold or platinum.

Therefore, the deposition on the deposition member can be started promptly.
(9) In the deposition status estimation apparatus according to the present invention, the etching solution is sodium persulfate (SPS) or nitric acid.

Therefore, the metal film can be etched without affecting the deposition member.
(10) A reaction container according to the present invention includes: a casing capable of holding a solution and having a plating solution inlet port, through which a plating solution is introduced from a plating tank, a plating solution outlet port, through which the plating solution is discharged, an etching solution inlet port, through which an etching solution is introduced, and an etching solution outlet port, through which the etching solution is discharged; and a deposition member configured to be fixed to the casing, deposit a metal film by the plating solution introduced into the casing, and be usable repeatedly by dissolving the metal film by the etching solution.

Therefore, the deposition member can be used repeatedly for the processing.
(11) In the reaction container according to the present invention, an upper portion of the deposition member is coated so as not to react with the plating solution.

Accordingly, even when a solution level of the plating solution rises or falls, an area of a reaction region of the deposition member that is in contact with the plating solution does not vary. Therefore, the stable deposition is performed.
(12) A deposition status estimation method according to the present invention includes: a step of collecting a portion of a plating solution in a plating tank; a step of immersing at least a part of a deposition member in the collected plating solution to form a metal film on the deposition member; a step of etching the deposition member, which is formed with the metal film, by an etching solution to dissolve the metal film; and a step of estimating a deposition status of the metal film in the plating tank on the basis of a metal concentration-related value of the etching solution, in which the metal film is dissolved.

Therefore, the deposition status can be estimated in real time even during processing in the plating tank.
(a) A deposition status estimation apparatus according to the present invention is a deposition status estimation apparatus including: a reaction container having a deposition member; a plating solution introduction mechanism that introduces a plating solution in a plating tank into the reaction container in order to immerse the deposition member in the plating solution and deposit a metal film; a measurement equipment that measures an amount of the metal film deposited on the deposition member; and a controller that executes control for measurement, in which
the controller controls to: introduce the plating solution into the reaction container by the plating solution introduction mechanism to deposit the metal film on the deposition member; and use the measurement equipment to measure the amount of the deposited metal film, and estimate a deposition status of the metal film in the plating tank on the basis of the amount of the metal film.

Therefore, the deposition status can be estimated in real time even during processing in the plating tank.
(b) In the deposition status estimation apparatus according to the present invention, the controller measures the amount of the metal film deposited on the deposition member on the basis of any of the following methods (1) to (8):(1) the metal film on the deposition member is dissolved by reverse electrolysis, and the amount of the metal film is calculated on the basis of a current required for this reverse electrolysis; (2) weight of the deposition member before and after formation of the metal film is measured, and the amount of the metal film is calculated on the basis of a difference therebetween; (3) a high-frequency current is flowed through the deposition member on which the metal film is deposited, and the amount of the metal film is calculated on the basis of an amount of an eddy current generated on a surface of the deposition member; (4) a magnetic circuit including the deposition member is formed, and the amount of the metal film is calculated on the basis of a change in magnetic resistance before and after the formation of the metal film; (5) the deposition member, on which the metal film is formed, is irradiated with an X-ray, and the amount of the metal film is calculated on the basis of an amount of an emitted fluorescent X-ray; (6) the deposition member, on which the metal film is formed, is irradiated with a beta ray, and the amount of the metal film is calculated on the basis of an amount of a backscattered beta ray; (7) masking is performed to generate a step at the time of forming the metal film, the masking is removed after the formation of the metal film, the step is measured, and the amount of the metal film is calculated on the basis of the step; and (8) the deposition member, on which the metal film is formed, is cut, a thickness of the metal film on a cross section of the deposition member is measured, and the amount of the metal film is calculated on the basis of the thickness of the metal film.

Therefore, the deposition rate can be estimated on the basis of the amount of the metal film.
(c) A reaction container according to the present invention includes: a casing capable of holding a solution and having: a plating solution inlet port through which a plating solution is introduced from a plating tank; and a plating solution outlet port through which the plating solution is discharged; and a deposition member configured to be fixed to the casing, deposit a metal film by the plating solution introduced into the casing, and be usable repeatedly by dissolving the metal film by an etching solution.

Therefore, the deposition member can be used repeatedly for the processing.
(d) In the reaction container according to the present invention, an upper portion of the deposition member is coated so as not to react with the plating solution.

Accordingly, even when a solution level of the plating solution rises or falls, an area of a reaction region of the deposition member that is in contact with the plating solution does not vary. Therefore, the stable deposition is performed.
(e) A deposition status estimation method according to the present invention includes: a step of collecting a portion of a plating solution in a plating tank; a step of immersing at least a part of a deposition member in the collected plating solution to form a metal film on the deposition member; and a step of measuring a deposition amount of the metal film formed on the deposition member to estimate a deposition status of the metal film in the plating tank on the basis of the deposition amount.

Therefore, the deposition status can be estimated in real time even during processing in the plating tank.
(f) A deposition status estimation apparatus according to the present invention is a deposition status estimation apparatus including: a reaction container having a deposition member; a plating solution introduction mechanism that introduces a plating solution in a plating tank into the reaction container in order to immerse the deposition member in the plating solution and deposit a metal film; a measurement equipment that measures a metal concentration-related value of the plating solution; and a controller that executes control for measurement, in which
the controller controls to: introduce the plating solution into the reaction container by the plating solution introduction mechanism to deposit the metal film on the deposition member; use the measurement equipment to measure the metal concentration-related value of the plating solution before or immediately after being introduced into the reaction container; use the measurement equipment to measure the metal concentration-related value of the plating solution after being introduced into the reaction container and depositing the metal film on the deposition member; and estimate a deposition status of the metal film in the plating tank on the basis of the metal concentration-related values measured by the pre-plating and the post-plating.

Therefore, the deposition status can be estimated even during processing in the plating tank.
(g) A deposition status estimation method according to the present invention includes: a first step of collecting a portion of a plating solution in a plating tank; a second step of measuring a metal concentration-related value of the collected plating solution; a third step of immersing at least a part of a deposition member in the collected plating solution to form a metal film on the deposition member; a fourth step of measuring the metal concentration-related value of the plating solution after the metal film is formed on the deposition member; and a step of estimating a deposition status of the metal film in the plating tank on the basis of the metal concentration-related value that is measured in the second step and the fourth step.

Therefore, the deposition status can be estimated even during processing in the plating tank.

The term “apparatus” is a concept that includes not only an apparatus including one computer but also an apparatus including a plurality of the computers connected via a network or the like. Thus, in the case where the means (or may be a portion of the means) in the present invention is divided into the plurality of the computers, these plural computers correspond to the apparatus.

The “program” is a concept that includes not only a program directly executable by a CPU but also a source-format program, a compressed program, an encrypted program, a program working with an operating system to implement a function thereof, and the like.

### BRIEF DESCRIPTION OF THE DRAWINGS

FIG. 1 is a functional configuration view of a deposition status estimation apparatus according to an embodiment of the present invention.

FIG. 2 illustrates a system configuration of the deposition status estimation apparatus.

FIG. 3 is a cross-sectional view of a reaction container 4.

FIG. 4 illustrates a hardware configuration of a controller 20.

FIG. 5 is a flowchart of a deposition status estimation program 48.

FIG. 6 is a flowchart of the deposition status estimation program 48.

FIG. 7 is a graph illustrating a relationship between a plating film thickness and a temperature.

FIG. 8 is a view illustrating an example in which a plurality of the reaction containers 4 is provided.

FIG. 9 is a functional configuration view of a deposition status estimation apparatus according to another example.

FIG. 10 illustrates a system configuration of the deposition status estimation apparatus.

FIG. 11 is a flowchart of the deposition status estimation program 48.

FIG. 12 is a flowchart of the deposition status estimation program 48.

FIG. 13 is a functional configuration view of a generalized deposition status estimation apparatus.

### DESCRIPTION OF EMBODIMENTS

### 1. Overall Configuration

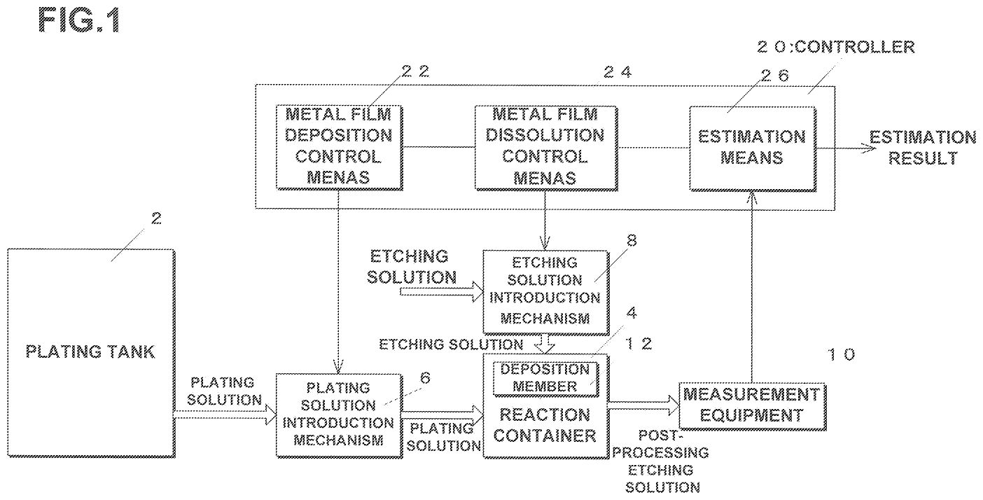

FIG. 1 illustrates a functional configuration of a deposition status estimation apparatus according to an embodiment of the present invention. Metal film deposition control means 22 of a controller 20 controls a plating solution introduction mechanism 6 to introduce a plating solution in a plating tank 2 into a reaction container 4. By the introduced plating solution, a metal film is deposited on a deposition member 12 that is provided in the reaction container 4. Next, metal film dissolution control means 24 of the controller 20 controls an etching solution introduction mechanism 8 to introduce an etching solution into the reaction container 4 instead of the plating solution. The introduced etching solution dissolves the metal film that is deposited on the deposition member 12.

A measurement equipment 10 measures a metal concentration-related value (metal concentration or a metal concentration-related value) of the post-processing etching solution in which the metal film is dissolved. Estimation means 26 of the controller 20 estimates a deposition status, such as a plating deposition rate, in the plating tank 2 on the basis of the metal concentration-related value measured by the measurement equipment 10.

As described above, a portion of the plating solution in the plating tank 2 is introduced into the reaction container 4, the metal is deposited on the deposition member 12, the deposited metal is dissolved, and the concentration of the deposited metal is measured, so as to estimate the deposition status such as a deposition amount and the deposition rate. Accordingly, the deposition status can be estimated in real time even when a plating process is performed in the plating tank 2.

### 2. System Configuration and Structure

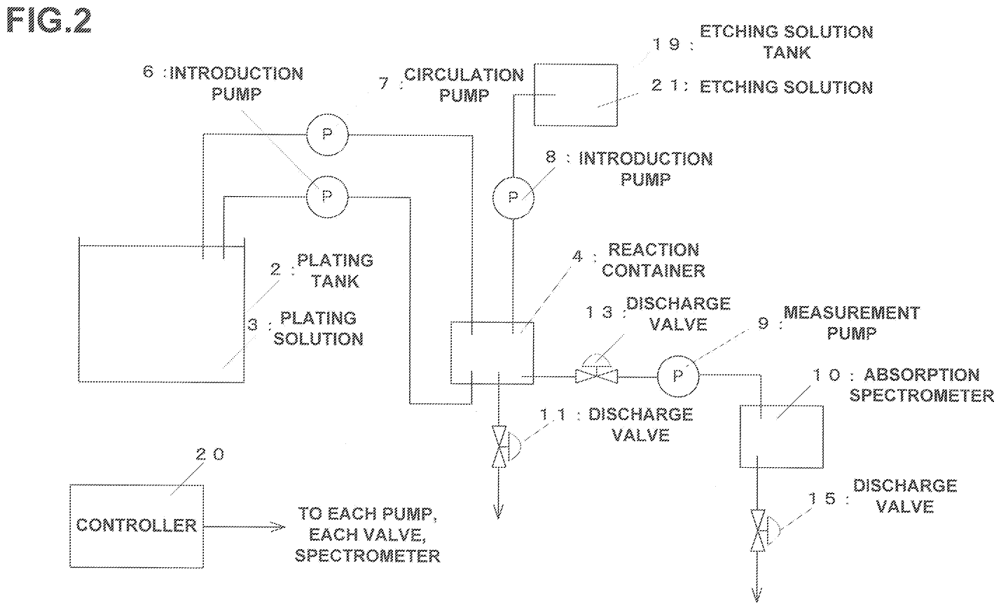

Hereinafter, a description will be made on electroless copper plating as an example. FIG. 2 illustrates a system configuration of the deposition status estimation apparatus. The plating tank 2 is a main tank for plating processing using a plating solution 3. A flow path is provided from the plating tank 2 to the reaction container 4 via an introduction pump 6 as the plating solution introduction mechanism. In addition, a flow path is provided from the reaction container 4 to the plating tank 2 via a circulation pump 7 for circulating the plating solution 3 in the reaction container 4 into the plating tank 2.

A flow path is provided from an etching solution tank 19 to the reaction container 4 via an introduction pump 8 as the etching solution introduction mechanism. A discharge valve 11 is provided to discharge the plating solution 3 or an etching solution 21 in the reaction container 4.

A flow path is provided from the reaction container 4 to an absorption spectrometer 10 as the measurement equipment via a discharge valve 13 and a measurement pump 9. A discharge valve 15 is provided to discharge the etching solution 21 in the absorption spectrometer 10. The etching solution preferably dissolves the metal film formed by plating but does not dissolve the deposition member 12. In this embodiment, sodium persulfate (SPS) and nitric acid are used as the etching solution.

The controller 20 controls each of the pumps and each of the valves, and receives output of the absorption spectrometer 10 to perform estimation processing.

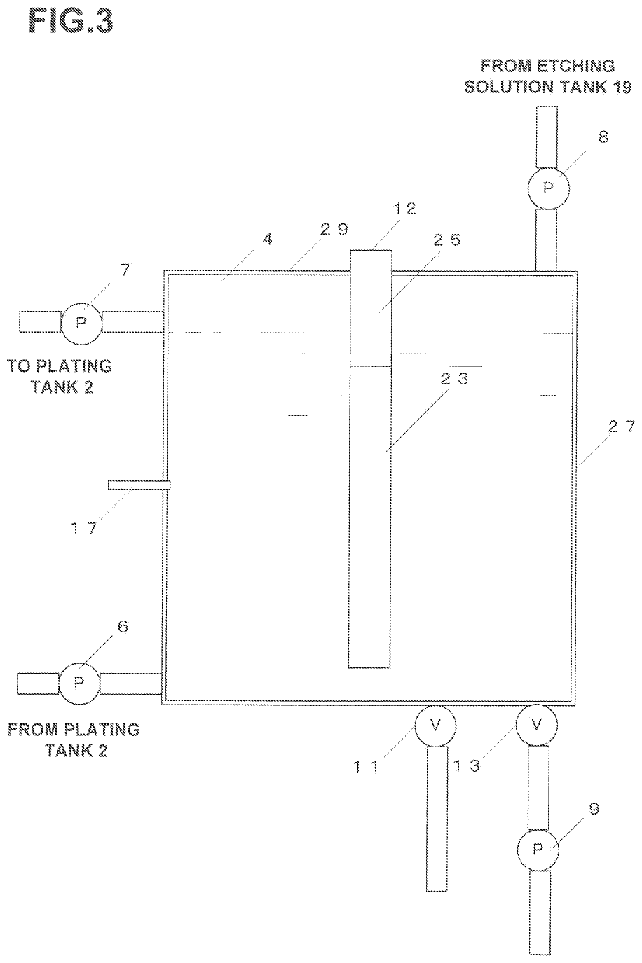

FIG. 3 illustrates a cross section of the reaction container 4. A lid 29 is provided to cover an upper opening of a bottomed cylindrical body 27. A columnar member 12 as the deposition member is fixed to the lid 29. The columnar member 12 is made of gold (Au) at least on a surface (may entirely be gold). An upper surface of the columnar member 12 has a coating 25 (for example, plating resist ink) to avoid a reaction with the plating solution. Thus, a reaction surface 23 is a region excluding this coating 25 portion.

The introduction pump 6 is provided to a lower portion of the reaction container 4 to introduce the plating solution 3 from the plating tank 2. The circulation pump 7 is provided to an upper portion of the reaction container 4 to circulate the plating solution 3 into the plating tank 2.

The introduction pump 8 is also provided to the upper portion of the reaction container 4 to introduce the etching solution from the etching solution tank 19. The discharge valve 13 is provided at a bottom of the reaction container 4, and the measurement pump 9 is provided to introduce the etching solution 21 in the reaction container 4 into the absorption spectrometer 10.

A temperature sensor 17 is provided to a central portion of the reaction container 4 to measure a temperature of the plating solution 3.

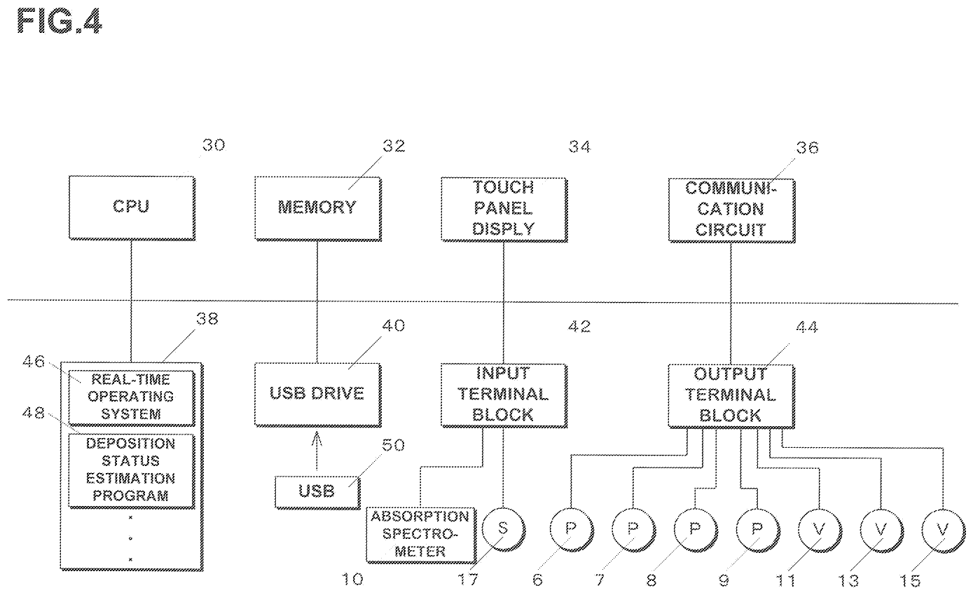

FIG. 4 illustrates a hardware configuration of the controller 20. Memory 32, a touch panel display 34, a communication circuit 36, flash memory 38, a USB drive 40, an input terminal block 42, and an output terminal block 44 are connected to a CPU 30. The communication circuit 36 is a circuit for connecting to a network such as a LAN or the Internet.

The absorption spectrometer 10 and the temperature sensor 17 are connected to the input terminal block 42. The CPU 30 acquires measurement data from the absorption spectrometer 10 and output of the temperature sensor 17 via the input terminal block 42.

The introduction pump 6, the circulation pump 7, the introduction pump 8, the measurement pump 9, and the discharge valves 11, 13, 15 are connected to the output terminal block 44. The CPU 30 controls these devices via the output terminal block 44.

A real-time operating system 46 and a deposition status estimation program 48 are recorded in the flash memory 38. The deposition status estimation program 48 cooperates with the real-time operating system 46 to implement functions thereof. These programs are recorded in a USB 50 and installed in the flash memory 38 via the USB drive 40.

### 3. Deposition Rate Estimation Processing

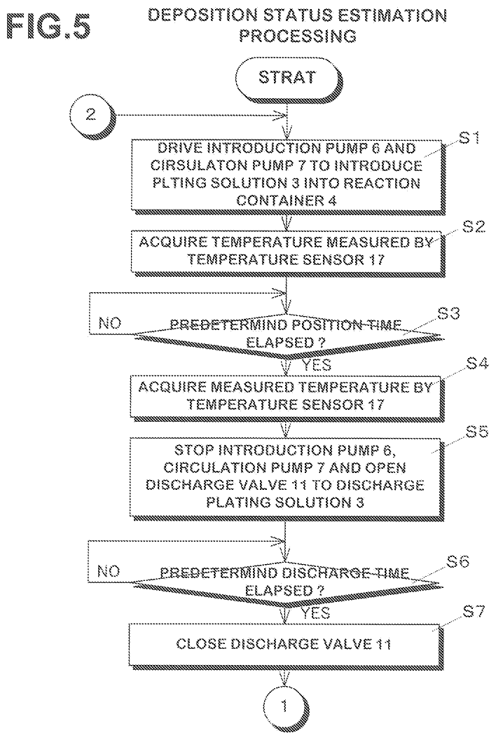

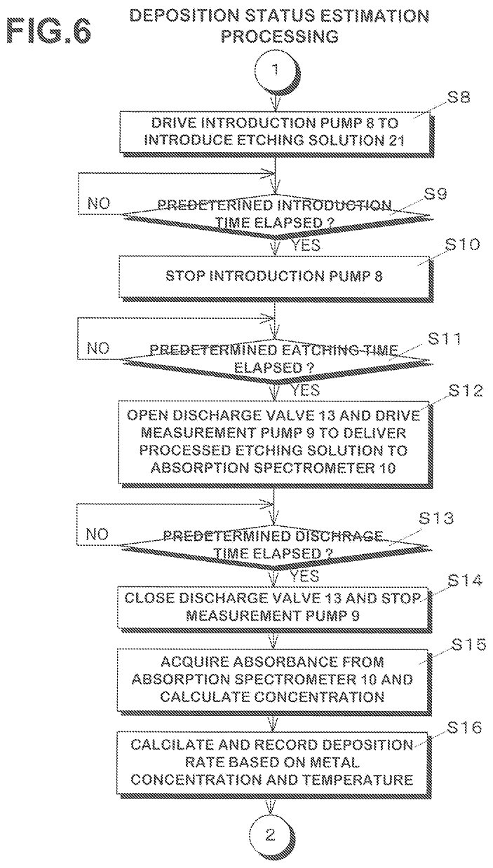

FIG. 5 and FIG. 6 illustrate flowcharts of the deposition status estimation program 48. The CPU 30 of the controller 20 drives the introduction pump 6 and the circulation pump 7 to introduce the electroless copper plating solution 3 in the plating tank 2 into the reaction container 4 (step S1). Next, the CPU 30 acquires the measured temperature data by the temperature sensor 17 that has measured the temperature of the electroless copper plating solution 3 introduced into the reaction container 4, and records the measured temperature data (step S2).

Since the reaction container 4 is provided with the gold columnar member 12, copper is deposited on the surface thereof by the electroless copper plating solution 3. Since the metal is deposited without pretreatment with gold, the deposition of copper is started immediately.

Since the electroless copper plating solution 3 is supplied from the plating tank 2 to the reaction container 4, and the electroless copper plating solution 3 is circulated into the plating tank 2, the new electroless copper plating solution 3 constantly contacts the columnar member 12 for stable deposition. In this embodiment, an amount of the electroless copper plating solution 3 that is held in the reaction container 4 is approximately 1 to 100 ml. In addition, since the temperature of the electroless copper plating solution 3 in the plating tank 2 is higher than a room temperature, the temperature may gradually be reduced. However, by circulating the electroless copper plating solution 3, the temperature reduction thereof can be suppressed.

Since the upper portion of the columnar member 12 has the coating 25, copper is not deposited on this portion. Accordingly, in the case where the uncoated reaction region 23 of the columnar member 12 is completely immersed in the electroless copper plating solution 3, an area in contact with the electroless copper plating solution 3 does not vary even when a solution level of the electroless copper plating solution 3 rises or falls slightly. Thus, copper can be deposited stably.

The CPU 30 keeps the above state for a predetermined time to continue the deposition of copper on the columnar member 12. When determining by an internal timer that the predetermined time (for example, 5 minutes to 30 minutes), which is set in advance for the deposition, has elapsed (step S3), the CPU 30 acquires and records the measured temperature data by the temperature sensor 17 again (step S4).

Next, the CPU 30 stops the introduction pump 6 and the circulation pump 7, opens the discharge valve 11, and discharges the electroless copper plating solution 3 from the reaction container 4 (step S5). When a sufficient time for the electroless copper plating solution 3 to be discharged from the reaction container 4 (a predetermined discharge time) elapses (step S6), the CPU 30 closes the discharge valve 11 (step S7). A level sensor may be provided to the reaction container 4 so as to enable the CPU 30 to detect completion of the discharge, or a flow meter may be provided to the discharge flow path of the discharge valve 11 to enable the CPU 30 to detect the completion of the discharge.

Next, the CPU 30 drives the introduction pump 8 and introduces the etching solution 21 into the reaction container 4 (step S8). When a sufficient time for the etching solution 21 to be filled in the reaction container 4 (a predetermined introduction time) elapses (step S9), the CPU 30 stops the introduction pump 8 (step S10). In this embodiment, the etching solution 21 of about 1 to 100 ml is introduced. The level sensor may be provided to the reaction container 4 so as to enable the CPU 30 to detect completion of filling, or the flow meter may be provided to the supply flow path of the introduction pump 8 to enable the CPU 30 to detect the completion of filling.

As described above, the etching solution 21 dissolves and etches all copper deposited in the reaction region 23 of the columnar member 12. In this way, deposited copper is dissolved in the etching solution 21, and concentration of copper in the etching solution 21 is increased in proportion to a deposition amount of copper. In addition, since copper deposited in the reaction region 23 of the columnar member 12 is removed, gold is exposed, and the columnar member 12 is restored to a state where the electroless copper plating can be performed again.

When detecting that a predetermined time (a predetermined etching time) (in this embodiment, 0.5 minute to 10 minutes) to allow complete dissolution of deposited copper by etching has elapsed (step S11), the CPU 30 opens the discharge valve 13 and drives the measurement pump 9 (step S12). In this way, the etching solution 21 in which copper is dissolved is delivered to the absorption spectrometer 10.

When a sufficient time for the etching solution 21 to be discharged from the reaction container 4 (a predetermined discharge time) elapses (step S13), the CPU 30 closes the discharge valve 13 (step S14). The level sensor may be provided to the reaction container 4 so as to enable the CPU 30 to detect completion of the discharge, or a flow meter may be provided to the discharge flow path of the discharge valve 13 to enable the CPU 30 to detect the completion of the discharge.

The absorption spectrometer 10 measures absorbance (the concentration-related value) of copper dissolved in the etching solution 21. The CPU 30 acquires the absorbance of copper from the absorption spectrometer 10. In general, a relationship between the absorbance and the concentration is known (linearly proportional). Thus, the CPU 30 calculates the concentration of copper (mol/m³) on the basis of the absorbance (step S15). After acquiring the absorbance of copper from the absorption spectrometer 10, the CPU 30 opens the discharge valve 15 to discharge the etching solution 21 from the absorption spectrometer 10.

Next, the CPU 30 calculates the deposition amount of copper on the basis of the copper concentration and a volume of the etching solution. Furthermore, the CPU 30 calculates the deposition rate (μm/min) of copper in the reaction container 4 on the basis of an area of the reaction region 23 of the columnar member 12, the deposition amount of copper, and the plating time in step S3.

The thus-calculated deposition rate is based on the temperature of the electroless copper plating solution 3 in the reaction container 4. As described above, the temperature of the electroless copper plating solution 3 in the reaction container 4 is lower than the temperature of the electroless copper plating solution 3 in the plating tank 2, which is the main tank.

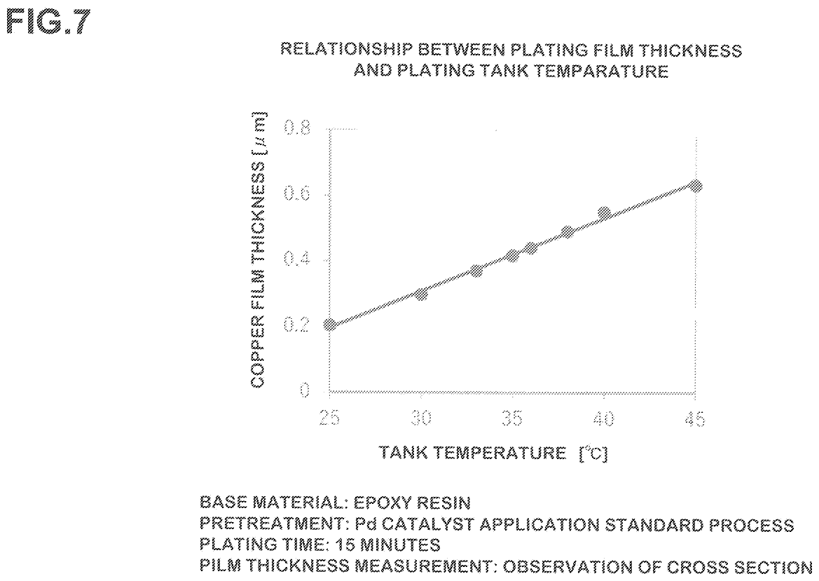

As illustrated in FIG. 7, a relationship between the temperature and the deposition rate (a film thickness) is known to be linear. Accordingly, the CPU 30 corrects the deposition rate, which has been calculated earlier, on the basis of a difference between the temperature of the electroless copper plating solution 3 in the plating tank 2 and the temperature of the electroless copper plating solution 3 in the reaction container 4, calculates the deposition rate in the plating tank 2, and records the deposition rate in the flash memory 38 (step S16). As it has been described so far, the deposition rate of copper in the plating tank 2 is estimated. The CPU 30 displays the estimated deposition rate on the touch panel display 34.

In the above estimation, as the temperature of the electroless copper plating solution 3 in the reaction container 4, an average of the temperatures (steps S2, S4) at the start and the termination of plating by the electroless copper plating solution 3 is used. In addition, as the temperature of the electroless copper plating solution 3 in the plating tank 2, a predetermined set temperature may be used, or the temperature may be acquired from a temperature sensor provided to the plating tank 2. In the latter case, the temperatures are preferably acquired at the same timing as steps S2, S4, and the average thereof is preferably used.

When the above processing is terminated, the CPU 30 executes step S1 onward again and repeats the introduction of the electroless copper plating solution 3, the deposition of copper, etching, the calculation of the concentration, and the calculation of the deposition rate. In this way, the deposition rate can be acquired in real time even during plating in the plating tank 2, which is the main tank.

By monitoring a change in the deposition rate, abnormality thereof can be detected (may be determined by an absolute value of the deposition rate or may be determined by a comparison with the normal deposition rate in the past) and can be notified by an alarm (not illustrated) or by display on the touch panel display 34. It is also possible to calculate the deposition rate prior to actual plating work and set the concentration of the plating solution, a required time, the temperature, and the like in the plating work.

### 4. Other Modified Examples

(1) In the above embodiment, the deposition rate is estimated as the deposition status in the plating tank 2. However, another deposition status such as a deposited film thickness may be estimated.
(2) In the above embodiment, the absorbance is used as the concentration-related value to be measured. However, another concentration-related value such as transmittance may be measured. The concentration may be measured as the concentration-related value.
(3) In the above embodiment, the columnar member is used as the deposition member. However, a member in another shape such as a prism may be used. In the above embodiment, gold is used as the deposition member. However, platinum or the like may be used.
(4) In the above embodiment, the description has been made on the case of the electroless copper plating. However, the present invention can also be applied to another metal such as electroless nickel.
(5) In the above embodiment, a heater is not provided to the reaction container 4. However, the heater may be provided to prevent a temperature reduction of the plating solution and to suppress the temperature difference from the plating solution 3 in the plating tank 2. In this case, the temperatures of the plating solution 3 in the plating tank 2 and the reaction container 4 may be measured by the sensors, and heating by the heater may be controlled such that the temperature of the plating solution 3 in the reaction container 4 becomes equal to the temperature of the plating solution 3 in the plating tank 2. In this case, the correction of the deposition rate due to the temperature difference is unnecessary.
(6) In the above embodiment, the temperature of the plating solution 3 is measured at the start of plating and at the termination of plating (steps S2, S4). However, the temperature of the plating solution 3 may only be measured at the start or the termination of plating, or the temperature may be measured only once between the start and the termination of plating. Alternatively, the temperature may be measured continuously from the start to the termination of plating, and an average value may be calculated.
(7) In the above embodiment, the circulation pump 7 is provided to return the plating solution 3 to the plating tank 2. However, the plating solution 3 may not be returned to the plating tank 2 and may be discharged.
(8) In the above embodiment, the plating solution 3 is supplied from the plating tank 2 during the deposition on the columnar member 12. However, the introduction pump 6 may be stopped at a time point when the reaction container 4 is filled with the plating solution 3.
(9) In the above embodiment, the columnar member 12 is held and fixed from above. However, the columnar member 12 may be fixed from below or a side. In this case, the coating 25 may not be provided as long as the columnar member 12 is completely immersed in the plating solution 3.
(10) In the above embodiment, the processed etching solution 21 is introduced into the absorption spectrometer 10 for the measurement. However, the absorption spectrometer 10 may be provided to the reaction container 4 for the measurement.
(11) In the above embodiment, the pump is used as the introduction mechanism. However, a valve or the like that controls natural fall of the solution may be used as the introduction mechanism.
(12) In the above embodiment, the metal film deposition control means 22, the metal film dissolution control means 24, and the estimation means 26 in FIG. 1 are implemented using software. However, some or all thereof may be configured by hardware logic.
(13) In the above embodiment, the processing in FIG. 5 and FIG. 6 is repeated to continuously estimate the deposition rate. However, in the case where time is required for the processing in FIG. 5 and FIG. 6, an estimation interval of the deposition rate is extended.

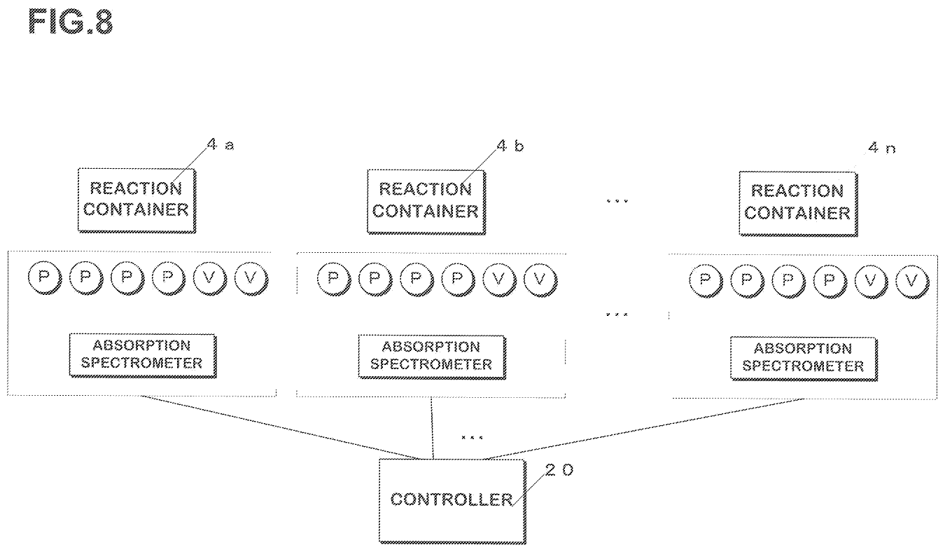

Accordingly, in the case where the deposition rate is required at the short estimation interval, as illustrated in FIG. 8, a plurality of the reaction containers may be provided. The controller 20 executes the control such that the deposition in a reaction container 4b is started (the introduction of the plating solution 3 is started) after a predetermined time from a start of the deposition in a reaction container 4a. Similarly, the controller 20 executes the control to sequentially start the deposition in the reaction containers 4c, 4d . . . 4n by delaying timing to start each deposition by the predetermined time. After the predetermined time from the start of the deposition in the reaction container 4n, the deposition in the reaction container 4a is started again. For this purpose, the processing in FIG. 5 and FIG. 6 in the reaction container 4a should be completed before the lapse of the predetermined time from the start of the deposition in the reaction container 4n.

In this way, the deposition rate can be estimated at the short intervals according to the number of the provided reaction containers.

In FIG. 8, the single controller 20 executes the control for the plurality of the reaction containers 4a to 4n. However, the controller 20 may be provided for each of the reaction containers 4a to 4n.
(14) In the above embodiment, the metal concentration (the absorbance) in the processed etching solution 21 is measured to estimate the deposition rate.

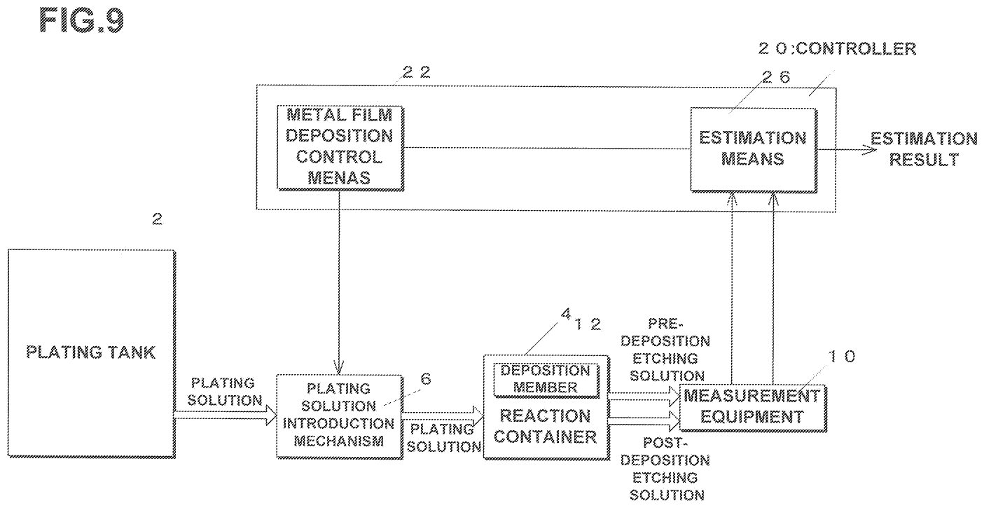

However, as illustrated in FIG. 9, the deposition rate may be estimated on the basis of a difference in the metal concentration in the plating solution before and after the deposition. In FIG. 9, the metal film deposition control means 22 of the controller 20 controls the plating solution introduction mechanism 6 to introduce the plating solution in the plating tank 2 into the reaction container 4. The measurement equipment 10 measures the metal concentration-related value of the plating solution at the time of the introduction, and provides the value to the estimation means 26.

By the introduced plating solution, the metal film is deposited on the deposition member 12 provided in the reaction container 4. The measurement equipment 10 measures the metal concentration-related value of the plating solution after the formation of the metal film, and provides the value to the estimation means 26.

The estimation means 26 of the controller 20 estimates the deposition status, such as the plating deposition rate, in the plating tank 2 on the basis of the metal concentration-related value before the formation of the metal film and the metal concentration-related value after the formation of the metal film, which are measured by the measurement equipment 10.

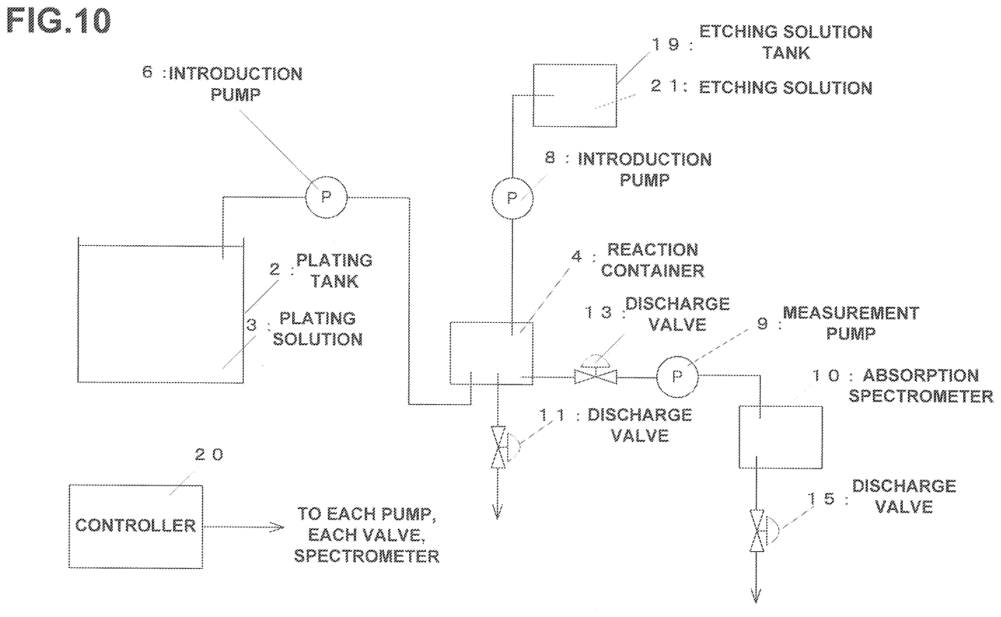

The system configuration in this case is as illustrated in FIG. 10. The plating solution 3 in the plating tank 2 is introduced into the reaction container 4 by the introduction pump 6. The controller 20 uses the absorption spectrometer 10 to measure the metal concentration in the plating solution 3 before the deposition and the metal concentration in the plating solution 3 after the deposition. The controller 20 estimates the deposition rate on the basis of the difference in the metal concentration between these two.

The basic structure of the reaction container 4, the basic hardware configuration of the controller 20, and the like are the same as those in the above embodiment.

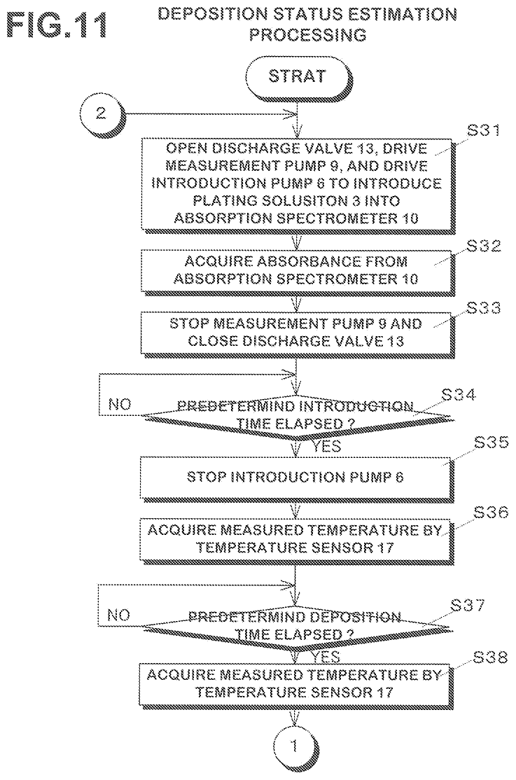

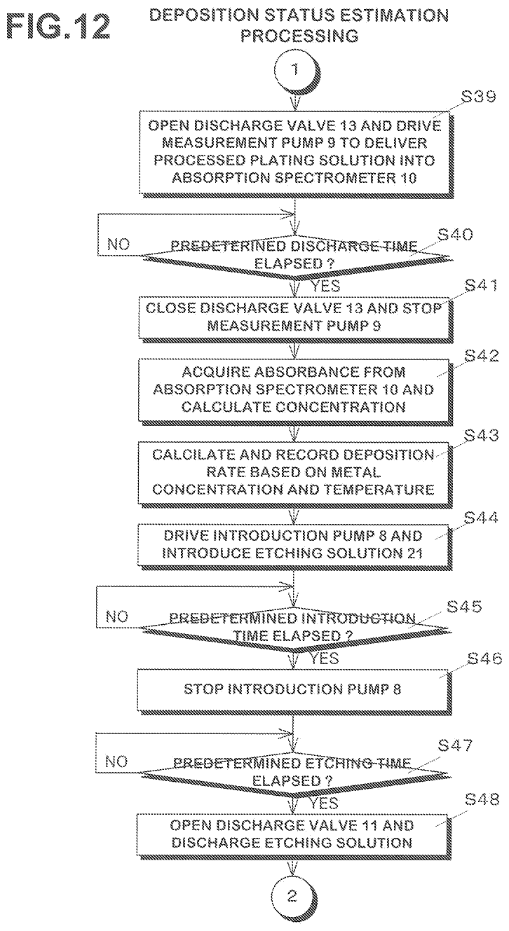

FIG. 11 and FIG. 12 illustrate flowcharts of the deposition status estimation program 48. The CPU 30 opens the discharge valve 13 and drives the introduction pump 6 and the measurement pump 9 to introduce the electroless copper plating solution 3 in the plating tank 2 into the absorption spectrometer 10 (step S31). The CPU 30 acquires and records the absorbance from the absorption spectrometer 10 at this time (step S32). Next, the CPU 30 closes the discharge valve 13 and introduces the electroless copper plating solution 3 into the reaction container 4 (steps S33, S34, S35). At this stage, the CPU 30 acquires and records the measured temperature by the temperature sensor 17 (step S36).

When the predetermined deposition time elapses (step, S37), the CPU 30 acquires the measured temperature by the temperature sensor 17. Then, the CPU 30 opens the discharge valve 13 and drives the measurement pump 9 to deliver the electroless copper plating solution 3 into the absorption spectrometer 10 (step S39).

The absorption spectrometer 10 measures the absorbance at this time, and the CPU 30 acquires the measured absorbance. The copper concentration in the electroless copper plating solution 3 after the deposition should be lower than the copper concentration in the electroless copper plating solution 3 before the deposition, by an amount of copper deposited on the columnar member 12. The CPU 30 calculates the deposition rate on the basis of the difference in the copper concentration in the electroless copper plating solution 3 before and after the deposition (steps S32, S42). In addition, the CPU 30 determines the temperature of the electroless copper plating solution 3 in the reaction container 4 as the average value between the temperature before the deposition (step S36) and the temperature after the deposition (step S38). Then, the CPU 30 corrects the deposition rate on the basis of the difference between this temperature and the temperature of the electroless copper plating solution 3 in the plating tank 2, and estimates the deposition rate in the plating tank 2 (step S43).

Thereafter, copper deposited on the columnar member 12 is removed by the etching solution 21, and the columnar member 12 is brought into a state where copper plating can be performed thereon again (steps S44 to S48).
(15) In the above embodiment, the deposition amount of the metal is calculated on the basis of the metal concentration in the processed etching solution.

However, the deposition amount of the metal can be calculated by another method.

For example, the metal film on the deposition member is dissolved by reverse electrolysis, and an amount of the metal film can be calculated on the basis of the current required for this reverse electrolysis. The amount of the metal film can be calculated by providing electrodes for the reverse electrolysis to the reaction container 4.

Weight of the deposition member 12 before and after the formation of the metal film is measured, and the amount of the metal film is calculated on the basis of the difference therebetween. This amount of the metal film can be calculated by fixing the deposition member 12 to the reaction container 4 via a digital weight scale. The weight is measured in a state where the reaction container 4 is not filled with the plating solution or the etching solution.

A high-frequency current is flowed through the deposition member 12 on which the metal film is deposited, and the amount of the metal film is calculated on the basis of an amount of an eddy current generated on the surface thereof. The amount of the metal film can be calculated by connecting a high-frequency power supply that generates the flow of the high-frequency current through the deposition member 12 and by providing an eddy current meter to measure the eddy current.

A magnetic circuit including the deposition member 12 is formed, and the amount of the metal film is calculated on the basis of a change in magnetic resistance before and after the formation of the metal film. The amount of the metal film can be calculated by providing a magnetic flux generator (such as an electromagnet) to form the magnetic circuit and providing a magnetoresistance measurement device.

The deposition member, on which the metal film is formed, is irradiated with X-rays, and the amount of the metal film is calculated on the basis of an amount of emitted fluorescent X-rays. The amount of the metal film can be calculated by providing an X-ray irradiator and a fluorescent X-ray measurement device.

The deposition member, on which the metal film is formed, is irradiated with beta rays, and the amount of the metal film is calculated on the basis of an amount of backscattered beta rays. The amount of the metal film can be calculated by providing a beta ray irradiator and a beta ray detector.

Masking is performed to generate a step at the time of forming the metal film, the masking is removed after the formation of the metal film, the step is measured, and the amount of the metal film is calculated on the basis of the step. The amount of the metal film can be calculated by providing a device for masking and a measurement device that measures the step.

The deposition member 12, on which the metal film is formed, is cut, the thickness of the metal film on a cross section of the deposition member 12 is measured, and the amount of the metal film is calculated on the basis of the thickness of the metal film.

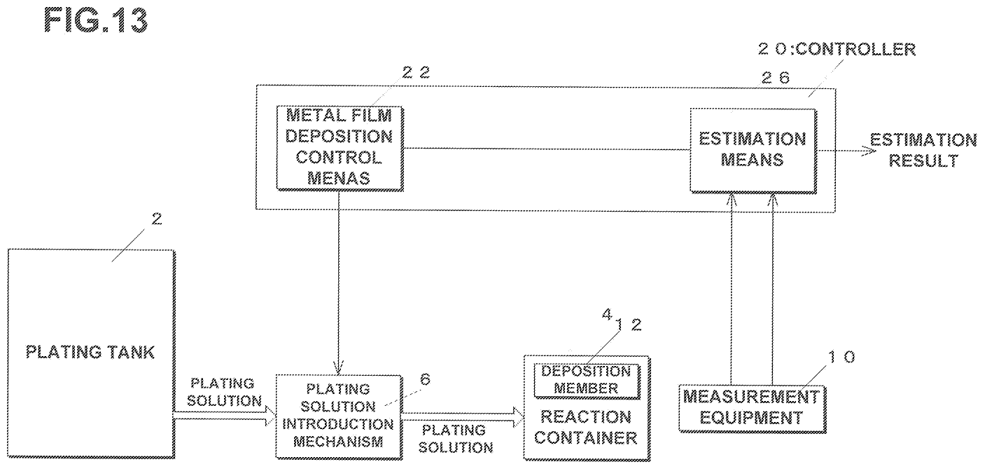

FIG. 13 illustrates a functional configuration that is obtained by abstracting what have been described so far. The measurement equipment 10 measures the amount of the metal film that is deposited on the deposition member 12 by the plating solution, and the estimation means 26 estimates the deposition rate in the plating tank 2.
(16) In the above embodiment, the estimated deposition rate and a warning are displayed on the touch panel display 34. Instead of or in addition to this, the communication circuit 36 may be used to output the deposition rate and the warning to another PC or a server device.
(17) Each of the above modified examples can be implemented in combination.

The subject matter described in the present invention is not restrictive and merely illustrative, and the claims are intended to include everything that falls within the scope of the present disclosure. To the fullest extent provided by the law, the scope of the present disclosure is specified by the broadest permissible interpretation of the following claims and their equivalents, which are incorporated into the detailed description herein, and shall not be limited or restricted by the aforementioned detailed description.

What is claimed is:

1. A deposition status estimation apparatus comprising:
a reaction container having a deposition member;
a plating solution introduction mechanism that introduces a plating solution in a plating tank into the reaction container in order to immerse the deposition member in the plating solution and deposit a metal film;
an etching solution introduction mechanism that introduces an etching solution into the reaction container in order to immerse the deposition member in the etching solution and dissolve the metal film;
a measurement equipment that measures a metal concentration-related value of the etching solution that has dissolved the metal film; and
a controller that executes control for measurement, wherein
the controller controls to:
introduce the plating solution into the reaction container by the plating solution introduction mechanism to deposit the metal film on the deposition member;
introduce the etching solution into the reaction container by the etching solution introduction mechanism after the plating solution in the reaction container is discharged, so as to dissolve the metal film deposited on the deposition member; and
use the measurement equipment to measure the metal concentration-related value of the etching solution, which has dissolved the metal film, and estimates a deposition status of the metal film in the plating tank on the basis of the metal concentration-related value.

2. The apparatus according to claim 1, wherein
when the metal film is deposited on the deposition member, the controller executes control to discharge the plating solution from the reaction container while introducing the plating solution in the plating tank into the reaction container.

3. The apparatus according to claim 1, wherein
when the metal film is deposited on the deposition member, the controller executes control to introduce the plating solution in the plating tank into the reaction container and stop the introduction once the deposition member is immersed.

4. The apparatus according to claim 1, wherein
the controller estimates a deposition rate in the plating tank as the deposition status.

5. The apparatus according to claim 1, wherein
the controller estimates the deposition status of the metal film in the plating tank in consideration of a difference between a temperature of the plating solution in the plating tank and a temperature of the plating solution in the reaction container, each of which is recorded or measured by a sensor.

6. The apparatus according to claim 1, wherein
the reaction container includes a heater that brings a temperature of the plating solution introduced into the reaction container close to a temperature of the plating solution in the plating tank.

7. The apparatus according to claim 1, wherein
the measurement equipment is provided in a path, through which the etching solution, which has dissolved the metal film, is discharged from the reaction container.

8. The apparatus according to claim 1, wherein
the metal film is copper or nickel, and
the deposition member is gold or platinum.

9. The apparatus according to claim 1, wherein
the etching solution is sodium persulfate (SPS) or nitric acid.

10. A reaction container comprising:
a casing capable of holding a solution and having: a plating solution inlet port through which a plating solution is introduced from a plating tank; a plating solution outlet port through which the plating solution is discharged; an etching solution inlet port through which an etching solution is introduced; and an etching solution outlet port through which the etching solution is discharged; and
a deposition member configured to be fixed to the casing, deposit a metal film by the plating solution introduced into the casing, and be usable repeatedly by dissolving the metal film by the etching solution.

11. The reaction container according to claim 10, wherein
an upper portion of the deposition member is coated so as not to react with the plating solution.

12. A deposition status estimation method comprising:
a step of collecting a portion of a plating solution in a plating tank;
a step of immersing at least a part of a deposition member in the collected plating solution to form a metal film on the deposition member;
a step of etching the deposition member, which is formed with the metal film, by an etching solution to dissolve the metal film; and
a step of estimating a deposition status of the metal film in the plating tank on the basis of a metal concentration-related value of the etching solution, in which the metal film is dissolved.
